(19) United States
(12) Patent Application Publication
(10) Pub. No.: US 2010/0191066 A1
(43) Pub. Date: Jul. 29, 2010

(54) SURGICAL PORT SYSTEM WITH MARKER RING

(76) Inventors: Scott Koysh, Lago Vista, TX (US); Robert J. Jones, Austin, TX (US)

Correspondence Address: Sprinkle IP Law Group / Zimmer;Suite 408, 1301 W. 25th Street, Austin, TX, 78705, US

(21) Appl. No.: 12/752,791

(22) Filed: Apr. 1, 2010

Related U.S. Application Data
(62) Division of application No. 11/303,349, filed on Dec. 16, 2005.

(51) Int. Cl. A61B 1/32; A61M 29/00

(52) U.S. Cl. 600/228; 600/201; 606/191

(57) ABSTRACT

Surgical port to maintain a working channel through bodily tissue includes a tubular body having a proximal portion and a distal portion, the tubular body having a working channel defined therethrough. Additionally, the surgical port includes a collar disposed at the proximal portion of the tubular body, the collar including a connection extending therefrom, and a radiopaque marker disposed at the distal portion of the tubular body. Dilatation system includes the surgical port as previously described, in combination with at least one tissue dilator.

### FIELD OF THE INVENTION

The present invention generally relates to retractor systems. A particular embodiment of the invention relates to a surgical port for establishing a working channel through bodily tissue, the surgical port including a radiopaque marker at a distal end thereof.

### BACKGROUND OF THE INVENTION

Tissue retractors are generally used during minimally invasive surgical procedures. In spinal surgeries, for example, a relatively small incision is made in the skin of a patient, and a retractor is inserted in the incision to form a working channel through bodily tissue to a surgical site. The working channel is dimensioned to allow a physician to manipulate one or more surgical tools therein to perform a surgical procedure. Upon completion of the surgical procedure, the retractor is removed and the incision closed. The minimally invasive procedure thus generally reduces trauma to the skin and tissue surrounding the surgical site.

Tissue retractors also can be used with a guide wire and one or more tissue dilators to dilate bodily tissue surrounding an incision before insertion of the retractor. For example, a guide wire is initially inserted within an incision formed in the skin of a patient. A first tissue dilator is positioned over the guide wire and inserted into the incision to the desired surgical site. As the first dilator advances into the incision, the tissue surrounding the surgical site is retracted or dilated radially outward. After insertion of the first dilator, one or more additional tissue dilators, each having a slightly larger inner diameter relative to the outer diameter of the previous dilator, can be inserted in the incision over the previously-inserted dilator if desired or needed. After the last dilator is inserted, a retractor or surgical port is inserted in the incision over the last dilator; the surgical port having an inner diameter larger than the outer diameter of the last dilator. The guide wire and dilator(s) can then be removed from the surgical port, for example, such that the retractor or surgical port forms a working channel through the bodily tissue to the surgical site.

Due to the relatively small size of the incision and related parts used for the procedure, the physician often is unable to visually inspect or confirm the position of the retractor or surgical port. Thus, there remains a need for a method and device to determine positioning of a surgical port within an incision.

### SUMMARY OF THE INVENTION

The purpose and advantages of the present invention will be set forth in and apparent from the description that follows, as well as will be learned by practice of the invention. Additional advantages of the invention will be realized and attained by the methods and systems particularly pointed out in the written description and claims hereof, as well as from the appended drawings.

The present invention is directed to a surgical port to maintain a working channel through bodily tissue. The surgical port of a preferred embodiment includes a tubular body having a proximal portion and a distal portion, the tubular body having a working channel defined therethrough. The surgical port also includes a collar disposed at the proximal portion of the tubular body, the collar including a connection extending therefrom, and a radiopaque marker disposed at the distal portion of the tubular body. Preferably, the radiopaque marker is made of a metallic material. More preferably, the metallic material is selected from the group consisting of stainless steel, iridium, platinum, tungsten, gold, barium, and tantalum. In one embodiment, the radiopaque marker is a wire member.

Preferably, the radiopaque marker is detachably engaged with the tubular body. In one embodiment, the distal portion of the tubular body has a groove defined therein, the groove being configured to receive the radiopaque marker therein. Preferably, the radiopaque marker is engaged by an interference fit within the groove.

In one embodiment, the tubular body of the surgical port is a single-piece member. Preferably, the working channel of the tubular body has an inner cross-dimension for receipt of a tissue dilator therethrough. Additionally, the distal portion of the tubular body preferably includes a tapered configuration. The tubular body and the collar can also be formed together as a single-piece member. Preferably, the connection of the collar includes a connecting arm.

The present invention is also directed to a dilation system to establish a working channel through bodily tissue. In one preferred embodiment, the dilation system includes at least one tissue dilator configured to dilate bodily tissue upon insertion thereof, the tissue dilator having an outer cross-dimension. The dilation system also includes a surgical port having a tubular body with a proximal portion and a distal portion, the tubular body having a working channel defined therethrough, a collar disposed at the proximal portion of the tubular body, the collar including a connection extending therefrom, and a radiopaque marker disposed at the distal portion of the tubular body.

Preferably, the dilation system includes a plurality of tissue dilators of increasing cross-dimensional size. The plurality of tissue dilators are configured for sequential dilation of the bodily tissue. In one embodiment, the working channel of the tubular body has an inner cross-dimension substantially similar to an outer cross-dimension of at least one of the plurality of tissue dilators. The dilation system also preferably includes a plurality of surgical ports, each surgical port including a tubular body of a different length and cross-dimension. In one embodiment, the length of each tubular body is between about 40 mm and about 100 mm.

The present invention is also directed to a method of dilating bodily tissue of a patient. A preferred embodiment of the method includes forming an incision through skin of a patient, inserting at least one tissue dilator through the incision, the tissue dilator having an outer cross-dimension, and positioning a surgical port through the incision over the at least one tissue dilator. The surgical port includes a tubular body having a proximal portion and a distal portion, the tubular body defining a working channel therethrough, and a radiopaque marker disposed at the distal portion of the tubular body. Preferably, the method further includes imaging the radiopaque marker to identify a location thereof.

In one embodiment, the method further includes sequentially inserting a plurality of tissue dilators of increasing cross-dimensional size through the incision. The surgical port preferably has an inner cross-dimension substantially similar to an outer cross-dimension of at least one of the plurality of tissue dilators.

Positioning of the surgical port can include selecting one of a plurality of surgical ports, each surgical port having a tubular body of a different length and cross-dimension. The surgical port is preferably supported on the skin of the patient surrounding the incision. After insertion of the surgical port, the tissue dilator is preferably removed from the incision.

In one embodiment, the surgical port further includes a collar disposed at the proximal portion of the tubular body, the collar having a connection arm extending therefrom, which can be connected to a support member to stabilize the surgical port. The tubular body of the surgical port also preferably defines an inner working channel for receipt of at least one instrument therethrough.

The present invention thus describes a surgical port that enables a physician to properly and accurately insert it through bodily tissue to the surgical site.

It is to be understood that both the foregoing general description and the following detailed description are exemplary and are intended to provide further explanation of the claimed invention.

The accompanying drawings, which are incorporated in and constitute part of this specification, are included to illustrate and provide a further understanding of the invention. Together with the description, the drawings serve to explain the principles of the invention.

### BRIEF DESCRIPTION OF THE DRAWINGS

FIG. 1 depicts an exploded view of an embodiment of a surgical port and marker member;

FIG. 2 depicts an assembled view thereof; and

FIG. 3 depicts a perspective view of an embodiment of a dilation system inserted through bodily tissue.

### DETAILED DESCRIPTION OF THE PREFERRED EMBODIMENTS

Reference will now be made in detail to a presently preferred embodiment of the invention, examples of which are illustrated in the accompanying drawings.

In accordance with the invention, a surgical port is provided for maintaining a working channel through bodily tissue. The surgical port includes a tubular body having a proximal portion and a distal portion. A working channel is defined through the tubular body. The surgical port further includes a collar disposed at the proximal portion of the tubular body. The collar includes a connection extending therefrom. Further in accordance with the invention, a radiopaque marker is disposed at the distal portion of the tubular body.

The surgical port is configured for use during minimally invasive surgical procedures. Various methods and related devices for such procedures include, for example, those described in U.S. patent application Ser. Nos. 10/697,793; 10/698,010; and 10/698,049, the contents of each hereby incorporated in its entirety by reference herewith.

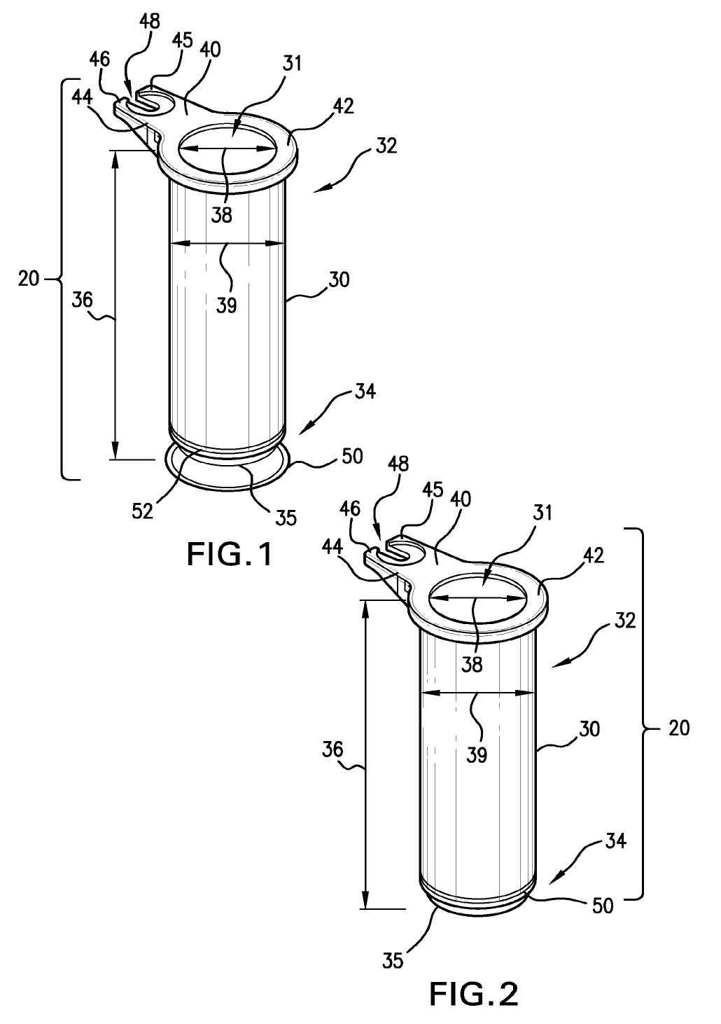

For purpose of illustration and not limitation, reference is made to the representative embodiment of the surgical port shown in FIGS. 1 and 2. A surgical port 20 is configured to maintain a working channel 31 through bodily tissue when inserted through an incision. The surgical port 20 includes a tubular body 30 that has a proximal portion 32 and a distal portion 34. As embodied herein, the surgical port also includes a collar 40 associated with the tubular body 30 at the proximal portion 32 thereof, and a radiopaque marker 50 associated with the tubular body 30 at the distal portion 34 thereof.

The tubular body 30 has a length 36, and the working channel 31 is defined through the tubular body 30, preferably along the entire length 36 thereof. The length 36 of the tubular body 30 is sufficient to enable the working channel 31 to extend at least from the skin of a patient to the surgical site, such as at the patient's spine. Generally, the length 36 of the tubular body 30 is preferably at least about 30 mm. In a preferred embodiment, such as for spinal procedures, the length 36 can be, for example, between about 40 mm and about 100 mm.

In one preferred embodiment, the tubular body 30 has a constant inner cross-dimension 38 and outer cross-dimension 39 along substantially the entire length 36 thereof. Alternatively, the inner cross-dimension and/or the outer cross-dimension can be varied along the length of the tubular body if desired. Additionally, the tubular body 30 can have a variety of cross-dimensional shapes, for example, circular, oval, or rectangular. Preferably, the tubular body is circular and the inner diameter 38 is sufficiently wide to accommodate a tissue dilator through the working channel 31. The inner cross-dimension 38 also is preferably is sufficiently wide to enable a physician to insert one or more surgical tools through the working channel 31 to perform a surgical procedure at the surgical site. Similarly, the outer cross-dimension 39 is preferably sufficiently small so as to minimize trauma imparted upon adjacent tissue when the surgical port is inserted through an incision. Preferably, the inner diameter 38 of the tubular body 30 is at least about 15 mm, although an inner diameter 38 between about 19 mm and about 26 mm is more preferred for most spinal procedures.

The distal portion 34 preferably has a tapered configuration such that a distal end 35 of the tubular body 30 has an outer cross-dimension smaller than the outer cross-dimension 39 of an adjacent portion of the tubular body. For example, in one embodiment having a circular cross-section, the distal portion has a substantially frustoconical configuration as shown in FIGS. 1 and 2. Advantageously, the tapered configuration of the distal portion 34 allows easy insertion of the surgical port 20 through an incision and minimizes the trauma to tissue that surrounds the surgical site.

As embodied herein, the tubular body 30 is made of a rigid material and has a unitary construction although it is recognized that a multi-member construction forming an adjustable retractor also can be provided. In one embodiment, for example, the tubular body 30 is made of a radiolucent material, such as RADEL® polysphenylsulfone. Advantageously, the radiolucent material of the tubular body 30 allows unimpeded visualization of the surgical site under fluoroscopy or other imaging techniques. In other embodiments, the tubular body 30 can be made of a radiopaque material, or other materials, such as metals, plastics, or composites.

The surgical port 20 also includes a collar 40 associated with the tubular body 30. Preferably, the collar 40 is associated with the tubular body 30 at the proximal portion 32 of the tubular body. With the collar 40 disposed at the proximal portion 32, the surgical port 20 retains a reduced profile above the incision when the tubular body 30 is inserted therein. In other embodiments, the collar 40 can be associated with the tubular body 30 at other locations along the length 36 thereof.

The collar can be provided in a variety of shapes, continuous or discontinuous. In one embodiment, the collar 40 includes an annular rim 42 that extends about the proximal portion 32 of the tubular body 30. Preferably, the annular rim 42 has an outer cross-dimension that is larger than the outer cross-dimension 39 of the tubular body 30. In this manner, the surgical port 20 can be fully inserted within the incision such that the collar 40 rests on the skin surrounding the incision. Additionally, the annular rim 42 in this configuration prevents over-insertion of the surgical port 20 within the incision. Alternatively, the collar can be formed by one or more protrusions extending from the tubular body.

The collar 40 includes a connection 44 extending outwardly therefrom. Preferably, the connection 44 is a connecting arm configured to associate with a surgical support arm, as is known in the art, to stablely support the surgical port 20 after insertion within an incision. In one embodiment, the connecting arm includes a pair of extending portions or tangs 45,46 that define a slot 48 therebetween. The connecting arm can thus connect or otherwise associate with a support arm for example, by threaded or interference fit connection.

The collar 40 is preferably made of a rigid material, such as a metal, plastic, or other composite. In a preferred embodiment, the collar is made of RADEL® polysphenylsulfone. Preferably, the collar 40 and tubular body 30 have a unitary construction. In other embodiments, the collar 40 and tubular body 30 are detachably engageable, for example, by a threaded or interference fit therebetween.

As previously noted, the surgical port includes a radiopaque marker, preferably disposed at the distal portion of the tubular body. The radiopaque marker 50 includes or is preferably made of a surgical grade metallic material. More preferably, the radiopaque marker 50 is made of stainless steel, iridium, platinum, tungsten, gold, barium, tantalum, or other radiopaque material. For example, the radiopaque market can be a coating or indicia applied to a surface of the tubular member, or one or more buttons or rivets attached at selected locations. In one embodiment, the radiopaque marker 50 is a ring member that can be disposed about the distal portion 34 of the tubular member 30. In other embodiments, the radiopaque marker 50 is a wire member, or similar member of low profile configuration. For example, a stainless steel wire with a diameter between about 0.039 inches and about 0.043 inches formed into a split ring configuration is preferred. The ends of the split ring are chamfered or otherwise processed to eliminate abrupt edges. A coated member can be used if desired.

Alternatively, the radiopaque marker 50 can be fixed to, or formed within, the tubular body 30. In a preferred embodiment, the radiopaque marker 50 is detachably engageable with the tubular body 30. In one embodiment, the distal portion 34 includes a groove 52 disposed about the outer surface of the tubular body 30. The groove 52 is preferably configured to receive the radiopaque marker 50 therein. More preferably, the radiopaque marker 50 is secured within the groove 52 by an interference fit. In this manner, for example, the radiopaque marker 50 is easily slipped over the distal portion 34 of the tubular body 30 and snapped into position within the groove 52 for simplified construction and selective use, as desired.

By associating the radiopaque marker 50 at the distal portion 34 of the tubular body 30, the physician can use fluoroscopy or other imaging techniques to visualize and confirm the position of the surgical port 20 relative to the surgical site. This advantageously allows the physician to position the surgical port 20 within an incision, and perform the surgical procedure through the working channel.

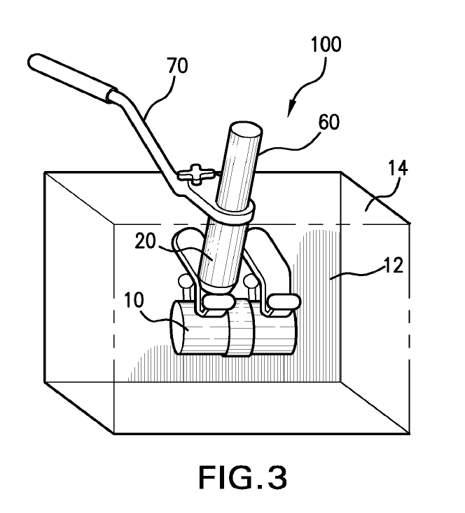

In accordance with another aspect of the invention, a dilation system is provided to establish a working channel through bodily tissue. The dilation system includes at least one tissue dilator configured to dilate bodily tissue upon insertion thereof, the tissue dilator having an outer cross-dimension. The dilation system also includes a surgical port having a tubular body that has a proximal portion and a distal portion. A working channel is defined through the tubular body. The surgical port also includes a collar disposed at the proximal portion of the tubular body, the collar including a connection extending therefrom, and a radiopaque marker disposed at the distal portion of the tubular body. As shown in FIG. 3, for example, a representative embodiment of the dilation system 100 includes the surgical port 20, as previously described, and at least one tissue dilator 60. In one embodiment, the tissue dilator 60 has an outer cross-dimension substantially similar to the inner cross-dimension 38 of the tubular body 30. In this manner, the surgical port 20 can be received over the tissue dilator 60 during use and insertion of the dilation system 100 within an incision.

Preferably, the dilation system 100 includes a guidewire and a set of tissue dilators 60, for example, three or four tissue dilators of increasing diameter configured to facilitate serial dilation of bodily tissue, as is known in the art. Serial dilation advantageously allows precise radial dilation of bodily tissue as desired, depending on the surgical procedure to be performed. Additionally, the dilation system can include a set of surgical ports, each surgical port having a tubular body of increasing length and/or cross-dimension. This combination of available ports allow selection of a proper surgical port for a particular surgical procedure, for example, depending on the distance from the incision to the surgical site and the diameter of the working channel that is required. In a preferred embodiment, at least one surgical port includes a radiopaque marked as previously described. Preferably, each surgical port includes a radiopaque marker.

As shown in FIG. 3, for purpose of illustration and not limitation, one embodiment of the dilation system 100 can be used to dilate bodily tissue 12 by first making an incision through the skin 14 of a patient. Preferably, the incision is made substantially directly over the surgical site 10, for example, a selected vertebrae or spinal structure. A guidewire preferably is then introduced through the incision to the surgical site 10. After introducing the guidewire, a first tissue dilator can be passed over the guidewire and into the tissue. The first dilator radically expands or dilates the bodily tissue to form a passage to the surgical site. Tissue dilators of increasingly larger diameter are then sequentially passed over the first dilator so as to further dilate the bodily tissue and form a sequentially larger insertion passage to the surgical site. Upon insertion of the largest dilator, the surgical port 20 can be positioned thereover within the incision, the surgical port 20 having an inner diameter substantially similar to outer diameter of the largest dilator. An appropriately-sized surgical port can be selected based on the length required to reach the surgical site or diameter of the largest dilator. Thus, the surgical port 20 establishes the final diameter of the passage and maintains the passage during a surgical procedure. The set of tissue dilators, including the guide wire, can be removed from the surgical port 20 to expose the working channel, and allow insertion and manipulation of surgical devices and instruments therethrough to the surgical site.

During positioning of the surgical port 20 within the incision and over the set of tissue dilators, a physician can advantageously identify the location of the surgical port 20 relative to the surgical site 10. The radiopaque marker located at the distal portion of the surgical port 20 is visible under fluoroscopy or other imaging techniques, thus enabling confirmation of a proper position, or allowing adjustment of the surgical port 20 to a proper position. Once proper positioning of the surgical port 20 is achieved, the surgical port can be stabilized within the incision. Preferably, the surgical port 20 is stabilized by securing the connecting arm of the collar to a support arm that is further secured, for example, to the operating table or other support. In one embodiment, the surgical port 20 is supported such that the tubular body can be fully inserted within the incision, with the collar resting on the skin surrounding the incision.

The term “about,” as used herein, should generally be understood to refer to both the corresponding number and a range of numbers. Moreover, all numerical ranges herein should be understood to include each whole integer within the range.

While illustrative embodiments of the invention are disclosed herein, it will be appreciated that numerous modifications and other embodiments can be devised by those of ordinary skill in the art. Features of the embodiments described herein can be combined, separated, interchanged, and/or rearranged to generate other embodiments. Therefore, it will be understood that the appended claims are intended to cover all such modifications and embodiments that come within the spirit and scope of the present invention.

1.-30. (canceled)

31. A minimally invasive surgical method, comprising:
making an incision substantially directly over a surgical site through skin of a patient;
introducing a guidewire to said surgical site through said incision;
passing one or more tissue dilators over said guidewire to form a passage from a dilated incision to said surgical site;
positioning a surgical port within said dilated incision to establish a final diameter of said passage, wherein said surgical port comprises:
a tubular body with a proximal end and a distal end;
a working channel within said tubular body and extending from said proximal end to said distal end, wherein said working channel is dimensioned to allow manipulation of one or more surgical tools therein;
a collar disposed at said proximal end of said tubular body and comprising a connection and an annular rim, wherein said connection extends from said proximal end of said tubular body and wherein said annular rim extends about the proximal end of the tubular body to prevent over-insertion of said surgical port within said dilated incision; and
a radiopaque marker disposed at said distal end of said tubular body; and
removing said one or more tissue dilators and said guidewire to expose said working channel of said surgical port.

32. The minimally invasive surgical method of claim 31, wherein positioning said surgical port within said dilated incision comprises selecting said surgical port from a plurality of surgical ports having different lengths and cross-dimensions.

33. The minimally invasive surgical method of claim 32, wherein said tubular body of said surgical port is between about 40 mm and about 100 mm in length.

34. The minimally invasive surgical method of claim 31, wherein said surgical port comprises an inner diameter, wherein said one or more tissue dilators comprise a last tissue dilator having an outer diameter, and wherein said inner diameter of said surgical port is substantially similar to said outer diameter of said last tissue dilator.

35. The minimally invasive surgical method of claim 31, further comprising identifying a location of said surgical port relative to said surgical site via said radiopaque marker disposed at said distal end of said tubular body of said surgical port.

36. The minimally invasive surgical method of claim 35, further comprising adjusting said location of said surgical port relative to said surgical site.

37. The minimally invasive surgical method of claim 31, further comprising stabilizing said surgical port within said dilated incision.

38. The minimally invasive surgical method of claim 37, wherein said stabilizing said surgical port within said dilated incision comprises:
securing said connecting of said collar of said surgical port to a support arm; and
securing said support arm to an operating table or structure.

39. The minimally invasive surgical method of claim 31, comprising maintaining said passage via said surgical port during a surgical procedure.

40. The minimally invasive surgical method of claim 31, wherein said surgical site is a portion of a spine or spinal structure.

41. The minimally invasive surgical method of claim 31, wherein said tubular body of said surgical port is a single-piece member.

42. The minimally invasive surgical method of claim 31, wherein said tubular body and said collar are formed together as a single-piece member.

43. The minimally invasive surgical method of claim 31, wherein said distal end of said tubular body includes a tapered configuration.

44. The minimally invasive surgical method of claim 31, wherein said distal end of said tubular body has a groove defined to receive the radiopaque marker therein.

45. The minimally invasive surgical method of claim 42, wherein said radiopaque marker is engaged by an interference fit within said groove.

46. The minimally invasive surgical method of claim 31, wherein said radiopaque marker is detachably engaged with said tubular body of said surgical port.

47. The minimally invasive surgical method of claim 31, wherein said radiopaque marker is a ring member.

48. The minimally invasive surgical method of claim 31, wherein the radiopaque marker is a wire member.
